(19) United States
(12) Patent Application Publication
(10) Pub. No.: US 2021/0015881 A1
(43) Pub. Date: Jan. 21, 2021

(54) AGENT EXHIBITING ANTIARRHYTHMIC EFFECT

(71) Applicant: Vladimir Vladimirovich SAMORODOV, Moscow (RU)

(72) Inventor: Vladimir Vladimirovich SAMORODOV, Moscow (RU)

(21) Appl. No.: 16/077,799

(22) PCT Filed: May 24, 2017
(86) PCT No.: PCT/RU2017/000341

§ 371 Date: Aug. 14, 2018

(30) Foreign Application Priority Data
Jun. 1, 2016 (RU) 2016121616

(51) Int. Cl. A61K 36/714; A61P 9/06; A61K 9/20; A61K 31/439

(52) U.S. Cl. CPC A61K 36/714; A61P 9/06; A61K 9/2013; A61K 31/439; A61K 9/2054; A61K 9/2059; A61K 9/2018

(57) ABSTRACT

The present invention comprises an agent exhibiting an antiarrhythmic effect, extracted from plants of genus Aconitum family Ranunculaceae, containing the alkaloids lappaconitine, N-acetylsepaconitine, 1-desmethyllappaconitine, ranaconitine, N-deacetyllappaconitine, isolappaconitine, 9-deoxy-lappaconitine or pharmaceutically acceptable salts thereof. The agent contains alkaloids in the form of hydrobromide salts of lappaconitine, N-acetylsepaconitine, 1-desmethyllappaconitine, ranaconitine, N-deacetyllappaconitine, isolappaconitine, and 9-deoxy-lappaconitine. In specific implementations, the agent may be extracted from the rhizomes and roots of the plant Aconitum septentrionale Koelle, from the rhizomes and roots of the plant Aconitum leucostomum, or from the herb of the plant Aconitum leucostomum, all of the family Ranunculaceae. A pharmaceutical composition for oral administration comprises the agent in an effective quantity and a pharmaceutically acceptable carrier. Preferably, the pharmaceutically acceptable carrier contains starch, sucrose, calcium stearate, and croscarmellose sodium. The present invention provides a more effective and safe cardioprotective agent for the prevention and treatment of arrhythmia.

### CROSS-REFERENCE TO RELATED APPLICATIONS

The present patent application National stage application of the PCT application PCT/RU2017/000341 filed May 24, 2017, which claims priority to Russian application RU 2016121616 filed Jun. 1, 2016.

### FIELD OF INVENTION

The invention relates to medicine, namely to chemical-pharmaceutical industry, in particular, to medicinal products and pharmaceutical compositions based on respective antiarrhythmic agents for correction of the myocardial functional state.

### BACKGROUND

There is a known antiarrhythmic drug Allapinin, which is a lappaconitine alkaloid hydrobromide (SU 1335293, prototype) made from northern wolfsbane (high wolfsbane).

The known medicinal product belongs to the membrane stabilizing medicinal products of I C class. The mechanism of action of the medicinal product is based on its ability to block rapid transmembrane voltage-dependent Na+ channels embedded into cardiomyocyte outer cell membrane and thus hinder the flow of Na+ ions into the cytosol of cardiomyocytes [A. E. Valeev et al. Neurophysiology. 1990. No. 2. pp. 201-206 (Ba [symbol] eeb A. E. c coabt. He [symbol] 1990 [symbol] o 2 C. 201-206]. It has been demonstrated that the medicinal product slows conduction of excitation and reduces the refractory period in atria, atrioventricular node, His bundle and Purkinje fibres [M. D. Mashkovsky. Medicinal products. M.: New wave, 2002. V. 1 P. 371 (Ma [symbol] M. [symbol] [symbol] cep [symbol] ctba. M.: H0Ba [symbol] BO [symbol] Ha, 2002. T. 1. C. 371)]. In clinical setting, the medicinal product is used at supraventricular and ventricular arrhythmia; flicker paroxysms and atrial flutter; paroxysmal supraventricular tachycardia (including the WPW syndrome); paroxysmal ventricular tachycardia (in the absence of organic lesions of the heart).

Allapinin belongs to 1 C class of medicinal products and is a highly effective antiarrhythmic agent for various forms of heart rhythm disorders and is particularly effective in the treatment of symptomatic benign ventricular arrhythmias (VA), paroxysmal atrial fibrillation and chronic unifocal atrial tachycardia.

Sudden cardiac death remains one of the major challenges in modern cardiology, including cardiopharmacology.

Reduced cardiac contractility, particularly in patients suffering from the coronary heart disease, especially if the disease is complicated by disturbance of the heart rhythm caused by ventricular fibrillation, is associated with a higher risk of sudden cardiac death. It has been demonstrated that in these patients the reduction of the cardiac ejection fraction, for example, from 40% to 30%, increases the risk of sudden cardiac death by 5 times [Santangeli P. et al. Hellenic J. Cardiol. 2007. V. 48. P. 72-79].

In addition, the phenomenon of arrhythmia is common in patients with the chronic obstructive pulmonary disease (COPD). The arrhythmia treatment with the existing medicinal products in these patients is complicated by the already existing high medicinal load on the organism. The COPD course in the patient may be accompanied by symptoms, which are typical for both COPD and arrhythmia in the presence of hypoxia. Should such a patient relieve the symptoms with medicinal products for COPD treatment, they may cause such side-effects as arrhythmia, which will aggravate the patient's state.

Currently, there are no data confirming the efficiency of Allapinin for the prevention of sudden cardiac death and treatment of arrhythmias in COPD patients. In this regard, it is urgent to search for an effective and safe medicinal product, which could be used in such patients.

### SUMMARY

A technical task of the invention is to provide for a safe and efficient cardioprotective means of arrhythmia prevention and treatment, sudden cardiac death risk reduction, treatment of arrhythmia in patients with the chronic obstructive pulmonary disease (COPD) and epilepsy, as well as to expand the range of cardioprotective agents that possess a high antiarrhythmic activity in various forms of arrhythmia.

The essence of the invention in the part of the medicinal product is that the antiarrhythmic agent obtained from Aconitum plants (wolfsbane) of Ranunculaceae family (buttercup family) contains alkaloids lappaconitine, N-acetylsepaconitine, 1-desmethyllappaconitine, ranaconitine, N-desacetyllappaconitine, isolappaconitine, 9-deoxylappaconitine or their pharmaceutically acceptable salts.

Usually the agent contains alkaloids in the form of hydrobromides of lappaconitine, N-acetylsepaconitine, 1-desmethyllappaconitine, ranaconitine, N-desacetyllappaconitine, isolappaconitine, 9-deoxylappaconitine.

In some cases of implementation, the agent can be isolated from rootstock of northern wolfsbane (high wolfsbane)-Aconitum septentrionale Koelle, buttercup family-Ranunculaceae.

In some cases of implementation, the agent can be isolated from rootstock of Aconitum leucostomum, buttercup family-Ranunculaceae.

In some cases of implementation, the agent can be isolated from herbage of Aconitum leucostomum, buttercup family-Ranunculaceae.

### DETAILED DESCRIPTION OF THE PREFERRED EMBODIMENT

The essence of the invention in the part of pharmaceutical composition is that the oral pharmaceutical composition contains the antiarrhythmic agent described by any of the above-mentioned cases of implementation in the effective quantity and the pharmaceutically acceptable carrier.

The pharmaceutically acceptable carrier should preferably contain starch, sucrose and calcium stearate, and, additionally, croscarmellose sodium.

The subject of the invention is a drug representing a pharmaceutical substance that consists of structurally related alkaloids isolated from plants of Aconitum genus (wolfsbane) belonging to Ranunculaceae family, such as northern wolfsbane (Aconitum septentrionale Koelle), whitethroat monkshood (Aconitum leucostomum), etc. All plants of this genus contain diterpenoid alkaloids. Both aerial part (herb) of the plant and rootstock may be used. The said pharmaceutical substance may be obtained in the following way.

A four-time extraction of alkaloids from the said raw material with ethyl alcohol (ethanol) is performed in a set of three extractors to obtain water-alcohol extracts, of which the first extract from each load of each extractor is used for evaporation, and the second, third and fourth extracts from each load are used as extracting agents for the first, second and third extracts from subsequent loads, with the fourth extraction from each load to be performed with ethanol. In the starting phase, the first, second and third portions of the raw material are loaded into the first, second and third extractors, respectively; the first extract from the first load is extracted with ethanol and sent to evaporation, the second extract from the first load is extracted with ethanol and sent to the second extractor to obtain the first extract from the second load and send it to evaporation, the third extract from the first load is extracted with ethanol and sent to the second extractor to obtain the second extract from the second load and send it to the third extractor in order to obtain the first extract from the third load and send it to evaporation, the fourth extract from the first load is extracted with ethanol and sent to the second extractor to obtain the third extract from the second load and send it to the third extractor in order to obtain the second extract from the third load and send it to the operational phase, the fourth extract from the second load is extracted with ethanol and sent to the third extractor to obtain the third extract from the third load and send it to the operational phase, the fourth extract from the third load is extracted with ethanol and sent to the operational phase, during which the fourth, fifth and sixth portions of the raw material are loaded into the first, second and third extractors, respectively; at the same time, the first extract from each load is sent to evaporation, and the second, third and fourth extract are used to obtain the first, second and third extracts from each subsequent load. The first water-alcohol extract is evaporated under vacuum, and the obtained aqueous evaporation bottoms are purified from ballast substances with ethyl acetate. The aqueous evaporation bottoms saturated with ethyl acetate are then acidified with mineral or organic acids, e. g. hydrobromic acid, and undergo multiple extractions with chloroform under controlled pH (not higher than 2), the chloroform extracts are evaporated under vacuum, and chloroform is displaced with ethanol to obtain the finished product suspension which is filtered, washed with ethanol, and dried.

Meanwhile, the first extraction of alkaloids from the first load of the starting phase is performed in the first extractor with 80% ethanol at 1:8 raw material/extracting agent ratio during 3 hours under room temperature and stirring, with subsequent recovery of ethanol from used raw material. The first extract is evaporated and purified from ballast substances four times, using water-saturated ethyl acetate during 30 minutes under stirring. The aqueous solution of the extract saturated with ethyl acetate is evaporated under vacuum, cooled down to room temperature, acidified with mineral or organic acids to pH not higher than 2, held up on a working mixer, and then treated with chloroform. The chloroform treatment is done four times to obtain four chloroform extracts that are evaporated under vacuum. Rectified ethanol is added to displace chloroform, and evaporated again until chloroform is removed completely.

The technical product prepared as described above or in other similar way contains alkaloids in a form of salts of the corresponding mineral or organic acid. The alkaloids content expressed as the corresponding lappaconitine salt is 87% to 94%. The technical product is dissolved in methanol under room temperature, and butanol is added (11% from the volume of the obtained solution). Then the obtained solution is evaporated under 91 to 95 kPa vacuum gauge pressure and temperature not higher than 70° C. until methanol is removed completely.

The resulting suspension is filtered and dried.

The pharmaceutical substance may also be obtained by other method.

The described method afforded the substance containing seven basic components specified in Table 1. Characteristics of the components' NMR spectra are given in Tables 2 and 3.

Minor components and impurities, whose content are hard to be determined quantitatively and depends on the specific type of used raw material, are also allowed.

The substance predominately contains lappaconitine (1). Associated alkaloids include N-acetylsepaconitine (2), 1-desmethyllappaconitine (3), ranaconitine (4), N-deacetyllappaconitine (5), isolappaconitine (6), 9-deoxylappaconitine (7). All components of the substance may be represented in a form of salts. The quantitative content of alkaloids varies between specific types and batches of used raw material.

TABLE 1

| No. | Common name | Formula | IUPAC name |
|---|---|---|---|
| 1 | Lappaconitine | 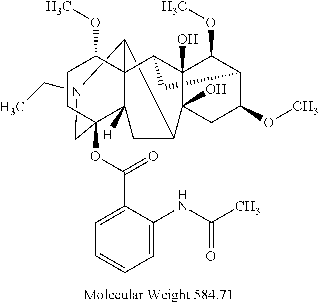 [SMILES: CCN1C[C@]2(OC(=O)c3ccccc3NC(C)=O)CC[C@H](OC)C34C1C(C[C@@H]32)[C@@]1(O)C[C@H](OC)[C@H]2C[C@@H]4[C@]1(O)[C@H]2OC] | (3S,6S,7S,7aS,8S,9R,10S, 11aS,14S)-1-ethyl-7a,11a- dihydroxy-6,8,10- trimethoxydodecahydro-2H- 3,6a,12- (epiethane[1,1,2]triyl)-7,9- methanonaphtho[2,3- b]azocine-3(4H)-yl 2- acetamidobenzoate |
| 2 | N-acetylsepaconitine | 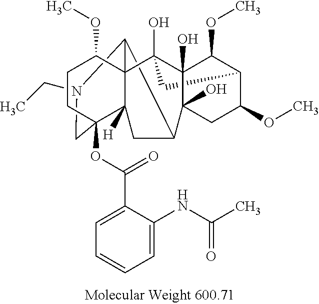 [SMILES: CCN1C[C@]2(OC(=O)c3ccccc3NC(C)=O)CC[C@H](OC)C34C1C(C[C@@H]32)[C@@]1(O)C[C@H](OC)[C@H]2C[C@]4(O)[C@]1(O)[C@H]2OC] | (3S,6S,7R,7aS,8S,9R,10S, 11aS,14S)-1-ethyl-7,7a,11a- trihydroxy-6,8,10- trimethoxydodecahydro-2H- 3,6a,12- (epiethane[1,1,2]triyl)-7,9- methanonaphtho[2,3- b]azocine-3(4H)-yl 2- acetamidobenzoate |
| 3 | 1-Desmethyllappaconitine | 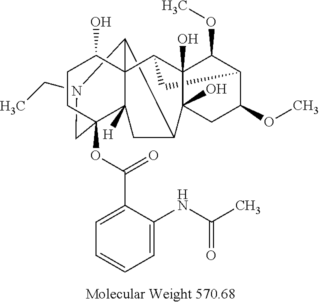 [SMILES: CCN1C[C@]2(OC(=O)c3ccccc3NC(C)=O)CC[C@H](O)C34C1C(C[C@@H]32)[C@@]1(O)C[C@H](OC)[C@H]2C[C@@H]4[C@]1(O)[C@H]2OC] | (3S,6S,7S,7aS,8S,9R,10S, 11aS,14S)-1-ethyl-6,7a,11a- trihydroxy-8,10- dimethoxydodecahydro-2H- 3,6a,12- (epiethane[1,1,2]triyl)-7,9- methanonaphtho[2,3- b]azocine-3(4H)-yl 2- acetamidobenzoate |
| 4 | Ranaconitine | 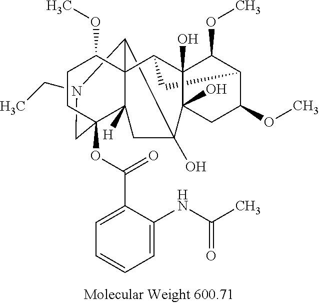 [SMILES: CCN1C[C@]2(OC(=O)c3ccccc3NC(C)=O)CC[C@H](OC)C34C1C(O)(C[C@@H]32)[C@@]1(O)C[C@H](OC)[C@H]2C[C@@H]4[C@]1(O)[C@H]2OC] | (3S,6S,7S,7aS,8S,9R,10S, 11aS,14S)-1-ethyl-7a,11a,12- trihydroxy-6,8,10- trimethoxydodecahydro-2H- 3,6a,12- (epiethane[1,1,2]triyl)-7,9- methanonaphtho[2,3- b]azocine-3(4H)-yl 2- acetamidobenzoate |
| 5 | N-deacetyllappaconitine | 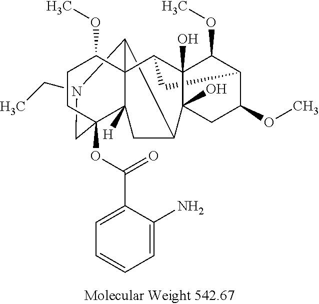 [SMILES: CCN1C[C@]2(OC(=O)c3ccccc3N)CC[C@H](OC)C34C1C(C[C@@H]32)[C@@]1(O)C[C@H](OC)[C@H]2C[C@@H]4[C@]1(O)[C@H]2OC] | (3S,6S,7S,7aS,8S,9R,10S, 11aS,14S)-1-ethyl-7a,11a- dihydroxy-6,8,10- trimethoxydodecahydro-2H- 3,6a,12- (epiethane[1,1,2]triyl)-7,9- methanonaphtho[2,3- b]azocine-3(4H)-yl 2- aminobenzoate |
| 6 | Isolappaconitine | 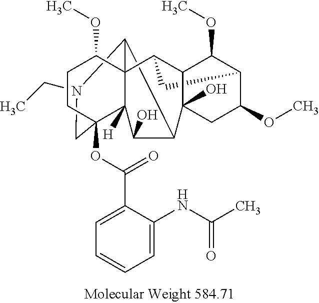 [SMILES: CCN1C[C@]2(OC(=O)c3ccccc3NC(C)=O)CC[C@H](OC)C34C1C([C@@H](O)[C@@H]32)[C@@]1(O)C[C@H](OC)[C@H]2C[C@@H]4C1[C@H]2OC] | (3S,6S,7R,8S,9R,10S,11aR, 13S,14R)-1-ethyl-11a,13- dihydroxy-6,8,10- trimethoxydodecahydro-2H- 3,6a,12- (epiethane[1,1,2]triyl)-7,9- methanonaphtho[2,3- b]azocine-3(4H)-yl 2- acetamidobenzoate |
| 7 | 9-Deoxylappaconitine | 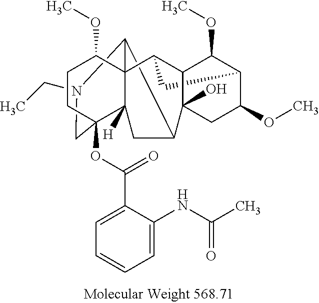 [SMILES: CCN1C[C@]2(OC(=O)c3ccccc3NC(C)=O)CC[C@H](OC)C34C1C(C[C@@H]32)[C@@]1(O)C[C@H](OC)[C@H]2C[C@@H]4C1[C@H]2OC] | (3S,6S,7R,8S,9R,10S,11aS, 14S)-1-ethyl-11a-hydroxy- 6,8,10- trimethoxydodecahydro-2H- 3,6a,12- (epiethane[1,1,2]triyl)-7,9- methanonaphtho[2,3- b]azocine-3(4H)-yl 2- acetamidobenzoate |

TABLE 2
Chemical shifts: ¹³C spectra.

| Lappaconitine | | 1-Desmethyllappaconitine | | Ranaconitine | | N-deacetyllappaconitine | |
|---|---|---|---|---|---|---|---|
| C1 | 85.4 | C1 | 72.1 | C1 | 83.9 | C1 | 84.4 |
| C2 | 28.0 | C2 | 30.3 | C2 | 27.1 | C2 | 26.3 |
| C3 | 32.9 | C3 | 29.9 | C3 | 31.9 | C3 | 32.0 |
| C4 | 86.1 | C4 | 82.7 | C4 | 84.8 | C4 | 82.9 |
| C5 | 49.4 | C5 | 43.9 | C5 | 50.9 | C5 | 48.8 |
| C6 | 25.2 | C6 | 27.3 | C6 | 32.3 | C6 | 26.9 |
| C7 | 49.5 | C7 | 46.5 | C7 | 86.9 | C7 | 47.7 |
| C8 | 76.0 | C8 | 76.2 | C8 | 76.5 | C8 | 75.7 |
| C9 | 80.1 | C9 | 77.5 | C9 | 78.4 | C9 | 78.6 |
| C10 | 52.3 | C10 | 48.3 | C10 | 36.3 | C10 | 49.9 |
| C11 | 52.5 | C11 | 50.3 | C11 | 50.9 | C11 | 51.0 |
| C12 | 27.4 | C12 | 23.6 | C12 | 26.3 | C12 | 24.1 |
| C13 | 37.7 | C13 | 36.3 | C13 | 48.7 | C13 | 36.4 |
| C14 | 91.3 | C14 | 90.3 | C14 | 89.7 | C14 | 90.2 |
| C15 | 44.5 | C15 | 45.3 | C15 | 36.0 | C15 | 44.8 |
| C16 | 84.7 | C16 | 82.8 | C16 | 83.6 | C16 | 83.0 |
| C17 | 62.2 | C17 | 63.3 | C17 | 62.2 | C17 | 61.6 |
| C19 | 55.9 | C19 | 58.0 | C19 | 55.0 | C19 | 55.7 |
| C21 (CH2) | 49.9 | C21 (CH2) | 48.3 | C21 (CH2) | 50.3 | C21 (CH2) | 49.1 |
| C22 (CH3) | 13.7 | C22 (CH3) | 13.0 | C22 (CH3) | 14.4 | C22 (CH3) | 13.6 |
| 1-OMe | 56.7 | | | 1-OMe | 55.9 | 1-OMe | 56.5 |
| 14-OMe | 58.2 | 14-OMe | 58.0 | 14-OMe | 57.5 | 14-OMe | 57.9 |
| 16-OMe | 56.4 | 16-OMe | 56.3 | 16-OMe | 56.3 | 16-OMe | 56.1 |
| C1′-C═O | 168.4 | C1′-C═O | 167.2 | C1′-C═O | 166.6 | C1′-C═O | 167.2 |
| C1′ | 118.7 | C1′ | 115.5 | C1′ | 118.5 | C1′ | 111.9 |
| C2′ | 141.9 | C2′ | 141.9 | C2′ | 140.5 | C2′ | 150.4 |
| C3′ | 122.0 | C3′ | 120.4 | C3′ | 121.5 | C3′ | 116.6 |
| C4′ | 135.1 | C4′ | 122.3 | C4′ | 123.5 | C4′ | 133.8 |
| C5′ | 124.2 | C5′ | 134.6 | C5′ | 134.3 | C5′ | 116.2 |
| C6′ | 132.1 | C6′ | 130.9 | C6′ | 131.1 | C6′ | 131.6 |
| N—C═O | 171.4 | N—C═O | 169.1 | N—C═O | 169.0 | N—C═O | — |
| CO—CH3 | 25.0 | CO—CH3 | 25.6 | CO—CH3 | 25.2 | CO—CH3 | — |

| N-acetylsepaconitine | | Isolappaconitine | | 9-Deoxylappaconitine | |
|---|---|---|---|---|---|
| C1 | 77.7 | C1 | 84.0 | C1 | 84.4 |
| C2 | 26.6 | C2 | 29.3 | C2 | 29.9 |
| C3 | 31.6 | C3 | 30.0 | C3 | 32.0 |
| C4 | 84.7 | C4 | 79.8 | C4 | 84.3 |
| C5 | 44.5 | C5 | 51.7 | C5 | 46.5 |
| C6 | 24.5 | C6 | 70.4 | C6 | 29.2 |
| C7 | 46.7 | C7 | 50.2 | C7 | 46.2 |
| C8 | 74.6 | C8 | 76.1 | C8 | 74.1 |
| C9 | 78.8 | C9 | 44.5 | C9 | 45.5 |
| C10 | 79.6 | C10 | 44.0 | C10 | 37.1 |
| C11 | 56.4 | C11 | 50.2 | C11 | 50.7 |
| C12 | 37.4 | C12 | 22.5 | C12 | 25.2 |
| C13 | 34.4 | C13 | 36.9 | C13 | 49.7 |
| C14 | 87.8 | C14 | 81.9 | C14 | 84.2 |
| C15 | 44.9 | C15 | 42.3 | C15 | 42.1 |
| C16 | 82.7 | C16 | 80.2 | C16 | 82.7 |
| C17 | 61.6 | C17 | 63.6 | C17 | 61.4 |
| C19 | 55.4 | C19 | 58.9 | C19 | 55.0 |
| C21 (CH2) | 48.9 | C21 (CH2) | 50.7 | C21 (CH2) | 48.9 |
| C22 (CH3) | 13.5 | C22 (CH3) | 10.1 | C22 (CH3) | 13.5 |
| 1-OMe | 56.2 | 1-OMe | 56.4 | 1-OMe | 56.6 |
| 14-OMe | 58.0 | 14-OMe | 57.8 | 14-OMe | 57.8 |
| 16-OMe | 56.3 | 16-OMe | 56.0 | 16-OMe | 56.2 |
| C1′-C═O | 167.4 | C1′-C═O | 166.7 | C1′-C═O | 167.7 |
| C1′ | 115.7 | C1′ | 115.9 | C1′ | 115.8 |
| C2′ | 141.7 | C2′ | 141.1 | C2′ | 141.9 |
| C3′ | 120.2 | C3′ | 121.1 | C3′ | 120.3 |
| C4′ | 134.4 | C4′ | 123.2 | C4′ | 122.4 |
| C5′ | 122.3 | C5′ | 135.1 | C5′ | 134.5 |
| C6′ | 131.0 | C6′ | 131.0 | C6′ | 131.2 |
| N—C═O | 169.1 | N—C═O | 169.6 | N—C═O | 163.3 |
| CO—CH3 | 25.6 | CO—CH3 | 25.3 | CO—CH3 | 25.6 |

TABLE 3
Chemical shifts: ¹H spectra.

| Lappaconitine | | 1-Desmethyllappaconitine | | Ranaconitine | | N-deacetyllappaconitine | |
|---|---|---|---|---|---|---|---|
| H1 | 3.26 dd (10.1; 7.2) | H1 | 3.82(3t) | H1 | 3.00 m | H1 | 3.20 (7&10 dd) |
| H2 | 2.20 m, 2.28 m | H2 | 2.33 m 2.42 m | H2 | 2.12 m | H2 | 2.01 m 2.53 m |
| H3 | 1.92 m, 2.60 m | H3 | 1.75 m 2.00 m | H3 | 1.85 m 2.53 m | H3 | 1.77 m2.72 m |
| H5 | 2.09 d (8.4) | H5 | 2.73 s | H5 | 1.89 m | H5 | 2.37 m |
| H6 | 1.54 m, 2.79 m | H6 | 1.78 m 2.20 m | H6 | 1.38(14 d) 2.99 m | H6 | 2.20 m 2.30 m |
| H7 | 2.40 d (7.5) | H7 | 2.19 m | | | H7 | 2.16(8 d) |
| H10 | 2.05 m | H10 | 2.17 m | H10 | 2.25 m | H10 | 2.01 m |
| H12 | 2.03 m, 2.45 m | H12 | 1.70 m 2.70 m | H12 | 1.89 m 2.32 m | H12 | 1.65 m 2.67 m |
| H13 | 2.37 dd (7.9, 4.7) | H13 | 2.52(5 t) | H13 | 2.18 m | H13 | 2.38(5 t) |
| H14 | 3.39 dd (4.8, 1.1) | H14 | 3.50(5 d) | H14 | 3.31(5 d) | H14 | 3.43(5 d) |
| H15 | 2.15 m, 2.22 m | H15 | 2.13 m 2.36 m | H15 | 1.73 m 2.71 m | H15 | 2.03 m 2.40 m |
| H16 | 3.29 m | H16 | 3.42 m | H16 | 3.12 m | H16 | 3.32 m |
| H17 | 3.02 s | H17 | 2.92 s | H17 | 2.68 s | H17 | 3.00 s |
| H19 | 2.45 d (11.4)3.56 | H19 | 2.60(12 d) | H19 | 3.06(11 d) | H19 | 2.55(11 d) |
| | d (11.4) | | 3.36(12 d) | | 3.47(11 d) | | 3.62(11 d) |
| H21 CH2 | 2.52 q (7.0) | H21 CH2 | 2.60(7 q) | H21 CH2 | 2.87 (7 q) | H21 CH2 | 2.54(7.2 q) |
| H22 CH3 | 11.2 t (7.0) | H22 CH3 | 1.20(7 t) | H22 CH3 | 1.00(7 t) | H22 CH3 | 1.24(7.2 t) |
| 1-OMe | 3.31 s | | | 1-OMe | 3.19 s | 1OMe | 3.30 s |
| 14-OMe | 3.37 s | 14-OMe | 3.43 s | 14-OMe | 3.26 s | 14-OMe | 3.42 s |
| 16-OMe | 3.29 s | 16-OMe | 3.35 s | 16-OMe | 3.18 s | 16-OMe | 3.32 s |
| H3′ | 8.42 d (8.8) | H3′ | 8.70(8 d) | H3′ | 8.30 (8d) | H3′ | 6.62(8.2 d) |
| H4′ | 7.52 dd (8.4, 7.3) | H4′ | 7.058 t) | H4′ | 7.16 (8 t) | H4′ | 7.23(8.2 t) |
| H5′ | 7.12 dd (8.0, 7.3) | H5′ | 7.53(8 t) | H5′ | 7.57 (8 t) | H5′ | 6.60(8.2 t) |
| H6′ | 7.93 d (8.0) | H6′ | 7.93(8 d) | H6′ | 7.85 (8 d) | H6′ | 7.78(8.2 d) |
| CO—CH3 | 2.19 s | CO—CH3 | 2.26 s | CO—CH3 | 2.12 s | CO—CH3 | — |
| NH | broad | NH | 11.07 bs | NH | broad | NH/NH2 | 5.67 bs |

| N-acetylsepaconitine | | Isolappaconitine | | 9-Deoxylappaconitine | |
|---|---|---|---|---|---|
| H1 | 3.29 m | H1 | 3.77(4.5 t) | H1 | 3.73 (4.5 t) |
| H2 | 2.15 m, 2.31 m | H2 | 2.20 m 2.63 m | H2 | 1.28 m |
| H3 | 2.06 m, 2.43 m | H3 | 1.57 m 2.15 m | H3 | 1.88 m 2.64 m |
| H5 | 2.10 d (8.4) | H5 | 2.75 s | H5 | 2.35 m |
| H6 | 1.64 m, 2.51 m | H6 | 4.68 (7 d) | H6 | 1.91 m 2.36 m |
| H7 | 2.45 d (7.5) | H7 | 2.56(7 d) | H7 | 2.15 m |
| | | H9 | 3.19 m | H9 | 1.92 m |
| H10 | — | H10 | 2.32 m | H10 | 2.39 m |
| H12 | 1.93 m, 2.64 m | H12 | 1.90 m 1.13 m | H12 | 1.80 m 2.07 m |
| H13 | 2.29 m | H13 | 2.53 m | H13 | 2.29 m |
| H14 | 3.28 dd (4.8, 1.1) | H14 | 3.25 m | H14 | 3.19 (7 d) |
| H15 | 2.18 m, 2.21 m | H15 | 2.16 m 2.30 m | H15 | 1.95 m 2.29 m |
| H16 | 3.30 m | H16 | 3.56 m | H16 | 3.21 m |
| H17 | 2.91 s | H17 | 3.58 s | H17 | 2.95 s |
| H19 | 2.58 d (11.4)3.57 d (11.4) | H19 | 2.82(13 d) 4.51(13 d) | H19 | 2.52(12 d) 3.56(12 d) |
| H21 CH2 | 2.58 q (7.0) | H21 | 3.07(7 q) | H21 CH2 | 2.55(7 q) |
| H22 CH3 | 1.14 t (7.0) | H22 | 1.41(7 t) | H22 CH3 | 1.14(7 t) |
| 1-OMe | 3.35 s | 1-OMe | 3.38 s | 1-OMe | 3.31 s |
| 14-OMe | 3.44 s | 14-OMe | 3.44 s | 14-OMe | 3.43 s |
| 16-OMe | 3.34 s | 16-OMe | 3.35 s | 16-OMe | 3.35 s |
| H3′ | 8.68 d (8.8) | H3′ | 8.49(8 d) | H3′ | 8.69(8 d) |
| H4′ | 7.52 t (8.4) | H4′ | 7.12(8 t) | H4′ | 7.05(8 t) |
| H5′ | 7.04 t (8.0) | H5′ | 7.56(8 t) | H5′ | 7.53(8 t) |
| H6′ | 7.92 d (8.0) | H6′ | 7.97(8 d) | H6′ | 7.94(8 d) |
| CO—CH3 | 2.25 s | CO—CH3 | 2.24 s | CO—CH3 | 2.25 s |
| NH | broad | NH | 10.5 bs | NH | 11.06 bs |

The pharmaceutical substance composition has been determined and described by means of a highly sensitive instrumental analysis method—high-performance liquid chromatography using a diode array detector and mass detector providing detection of both retention times and molecular weights of the components (hereafter, the component detection procedure).

The above mentioned parameters ensure high accuracy of identification for all seven basic components constituting the claimed drug. In addition, the procedure may be used to determine relative mass content of each component. Table 4 shows characteristics of the pharmaceutical substance components.

TABLE 4
Characteristics of the pharmaceutical substance components

| | | | Relative | |
|---|---|---|---|---|
| | | | content | Mole- |
| | | Relative | range, | cular |
| Item | | retention | % by | ion |
| No. | Alkaloid name | time range | weight | weight |
| 1. | Lappaconitine | 0.95 to 1.03 | 70.00 to 96.50 | 584.71 |
| 2. | N-acetylsepaconitine | 1.220 to 1.350 | 0.41 to 21.60 | 600.71 |
| 3. | 1- | 1.487 to 1.705 | 0.12 to 0.80 | 571.69 |
| | Desmethyllappaconitine | | | |
| 4. | Ranaconitine | 0.795 to 0.862 | 0.83 to 5.90 | 600.71 |
| 5. | N- | 1.734 to 1.904 | 1.00 to 4.50 | 542.67 |
| | deacetyllappaconitine | | | |
| 6. | Isolappaconitine | 1.430 to 1.550 | 0.25 to 1.20 | 585.72 |
| 7. | 9-Deoxylappaconitine | 2.000 to 2.200 | 0.15 to 0.42 | 568.71 |

The claimed drug is available in various oral dosage forms, such as tablets, capsules, granules, etc.

These forms may be obtained by known methods using known excipients typically applied in pharmaceutical industry.

Excipients used in solid oral formulations normally include fillers or diluents, binders, disintegrating agents, lubricants, anti-adhesives, glidants, moistening agents and surfactants, colorants, flavors, sweeteners, adsorbents, and organoleptic enhancers.

Fillers or diluents are added to an active substance in order to increase a tablet's volume. They include crystalline or amorphous lactose. Other diluents may be sugars, such as sucrose. Diluents and fillers are also represented by starch and its derivatives. Other starches include pregelatinized starch and sodium starch glycolate. Starches and their derivatives may be used as disintegrating agents. Other diluents include inorganic salts, such as dicalcium phosphate, tricalcium phosphate, and calcium sulphate. Other possible diluents are polyols, such as mannitol, sorbitol and xylitol. Many diluents also act as disintegrating agents and binders, and these additional properties must be taken into account when developing a formulation. Disintegrating agents are added to tablet formulations to ensure tablet disintegration into the particles of active pharmaceutical ingredient and excipients, thus improving the active ingredient solubility and increasing its bioavailability. Disintegrating agents include starch, gelatinized starch, croscarmellose sodium, crospovidone, microcrystalline cellulose, etc.

Binders are used in wet granulation. A binder functions as a powder flow enhancer and improves compressibility. Binders are cellulose derivatives, such as microcrystalline cellulose, methyl cellulose, sodium carboxymethyl cellulose, hydroxypropyl methyl cellulose, hydroxyethyl cellulose, and hydroxypropyl cellulose. Other binders include polyvinylpyrrolidone, gelatine, gum derivatives, pregelatinized starch, sucrose, polyethylene glycols, sodium alginate, etc.

Lubricants are used in tablet formulations to prevent adhesion of tablets to an impact surface, as well as to reduce friction during compression stages. Lubricants typically include vegetable oils, such as corn oil; mineral oils; polyethylene glycols; stearic acid salts, such as calcium stearate, magnesium stearate and sodium stearyl fumarate; mineral salts, such as talc; inorganic salts, such as sodium chloride; organic salts, such as sodium benzoate, sodium acetate and sodium oleate; and polyvinyl alcohols.

Glidants are used in solid dosage forms as flow aids for a bulk material to be tableted. The substances typically used for this purpose are as follows: microcrystalline cellulose; alkali metal stearates, such as magnesium stearate or calcium stearate; silicate salts such as magnesium silicate or calcium silicate; starch and starch variations and/or their derivatives; talc; colloidal silica, e. g., well-known Aerosil®.

The oral dosage form should preferably contain an effective quantity of the claimed drug and a pharmaceutically acceptable carrier containing sucrose, starch, and calcium stearate. According to another embodiment, the carrier additionally includes croscarmellose sodium.

The technical result is the creation of an efficient and safe cardioprotective agent for the prevention and treatment of arrhythmia, possessing the expanded possibilities of prevention of potentially malignant and malignant ventricular arrhythmias, ventricular fibrillation and reduction of the ejection fraction (index of myocardial contractility), which is the immediate cause of sudden cardiac death, as well as new functionalities of the complex treatment of arrhythmia, complicated by the chronic obstructive pulmonary disease (COPD) and epilepsy, as well as the expansion of the range of cardioprotective agents that possess a high antiarrhythmic activity in various forms of arrhythmia.

The achieved technical result was verified in accordance with the examples set forth below.

### EXAMPLE 1

As a result of implementation of the production method of the above-mentioned specified agent, pharmaceutical substances 1, 2, 3, 4, 5, 6, 7, 8, 9 were obtained, which contain alkaloid hydrobromides: lappaconitine, N-acetylsepaconitine, 1-desmethyllappaconitine, ranaconitine, N-desacetyllappaconitine, isolappaconitine, 9-deoxylappaconitine, the relative content of which is presented in Table 5.

TABLE 5

| | Relative content, % by weight | | | |
|---|---|---|---|---|
| | Ranaconitine | N-acetylsepaconitine | Isolappaconitine | 1-Desmethyllappaconitine |
| Substance 1 | 5.623 | 11.643 | 0.831 | 0.465 |
| Substance 2 | 5.828 | 12.647 | 0.785 | 0.329 |
| Substance 3 | 0.846 | 0.426 | 1.023 | 0.141 |
| Substance 4 | 2.19 | 7.492 | 0.553 | 0.693 |
| Substance 5 | 1.311 | 4.634 | 0.691 | 0.419 |
| Substance 6 | 2.141 | 17.645 | 0.306 | 0.519 |
| Substance 7 | 2.167 | 17.226 | 0.289 | 0.491 |
| Substance 8 | 2.453 | 21.5 | 0.366 | 0.777 |
| Substance 9 | 1.402 | 0.568 | 1.129 | 0.162 |
| | Relative content, % by weight | | | |
| | N-deacetyllappaconitine | 9-Deoxylappaconitine | Lappaconitine | Impurities |
| Substance 1 | 4.436 | 0.238 | 75.400 | 1.364 |
| Substance 2 | 3.299 | 0.135 | 75.868 | 1.109 |
| Substance 3 | 1.109 | 0.168 | 96.180 | 0.107 |
| Substance 4 | 3.403 | 0.139 | 85.167 | 0.363 |
| Substance 5 | 2.479 | 0.399 | 89.619 | 0.448 |
| Substance 6 | 3.147 | 0.293 | 74.926 | 1.023 |
| Substance 7 | 2.673 | 0.242 | 76.123 | 0.789 |
| Substance 8 | 2.995 | 0.264 | 70.596 | 1.049 |
| Substance 9 | 1.555 | 0.384 | 94.582 | 0.218 |

### EXAMPLE 2

### Preclinical Study Results

### Impact on the Development of Aconitic Arrhythmia

In control experiments, performed in 30 anesthetized rats, intravenous administration of aconitine at a dose of 20 μg/kg in 100% of cases caused allorythmic extrasystolic arrhythmia lasting from 59 to 156 minutes.

In order to create an aconitic arrhythmia model, the animals received aconitine intravenously 10 minutes after the intravenous administration of the studied substance. The experiment in 40 white outbred rats showed the antiarrhythmic effect of the studied substances at a dose of at least 0.028 μg/kg. At doses of 0.23-1.05 mg/kg, the subject substances prevented aconitic arrhythmia in 100% of animals.

Thus, intravenous administration of the studied substances prevented the occurrence of various arrhythmias and cardiac conduction caused by the administration of aconitine, which demonstrates a pronounced preventive antiarrhythmic effect of the studied substances.

Antiarrhythmic Activity in the Aconitic Arrhythmia model

The study was conducted in 50 white outbred rats. Aconitine was administered intravenously at a dose of 15-20 μg/kg and 5 minutes later the studied substance was administered intravenously in the background of the developed arrhythmia. The study revealed that substances 1-9 had a pronounced antiarrhythmic effect at doses of 0.34-0.50 mg/kg, which leads to a complete elimination of the arrhythmia and restoration of the normal sinus rhythm within 20 minutes after administration of the studied substances.

### Impact on the Lethal Heart Fibrillation Induced by Aconitine in Rats

The study included control experiments, in the course of which administration of aconitine to intact rats at a dose of 40 μg/kg caused ventricular extrasystole after the average time of 23.5±5.6 seconds, which 10-15 minutes later was transformed into 100% lethal atrial flutter and ventricular fibrillation.

The preventive administration of the studied substances in rats at a dose of 0.2 mg/kg demonstrated a 60% (6 out of 10) survival rate, which suggests that substances 1-9 prevent the development of lethal heart fibrillation.

The preventive administration of the studied substances in rats at doses of 1-2 mg/kg demonstrated a 100% (10 out of 10) survival rate, indicating that in this range of doses the studied substances 1-9 exhibit a pronounced antifibrillation effect and completely prevent lethal atrial fibrillation.

### Duration of Antiarrhythmic Effect

The study was conducted in 60 white outbred rats. Solutions of the studied substances were administered intragastrically at doses of 2 and 4 mg/kg 2, 4, 6, 8, 10, 12, 24 hours prior to the administration of aconitine. The study demonstrated that intragastric administration of substances 1-9 in both studied doses 2, 4, 6, 8, 10, 12, 24 hours prior to the introduction of aconitine prevented the arrhythmogenic effect of aconitine.

Thus, it has been revealed that the antiarrhythmic effect of substances 1-9 is maintained for 24 hours.

### Effect on the Threshold of Electrical Fibrillation of Ventricles in Rats

The study was conducted in 20 white outbred rats weighing 350-400 g. The animals were randomized into 2 groups: control (n=10) and animals, which received intravenous injections of the studied substance (n=10). The animals were intubated, then converted to artificial respiration and procedures of sternectomy and pericardiotomy were performed. Two gold-plated electrodes were implanted into the left ventricular myocardium at a distance of 0.5 cm from each other. The threshold of electrical fibrillation of the heart was determined by repeated scanning of the vulnerable period of the cardiac cycle in a series of 20 direct current square pulses of increasing intensity (stimulus duration was 4 msec, the frequency was 50 pulses/sec). The threshold of ventricle fibrillation was defined as the minimum current amplitude leading to ventricle fibrillation if repeated twice. Only those animals were included, in which the ventricle fibrillation occurred at the current amplitude of no more than 6 mA. ECG was registered throughout the experiment (the II standard lead).

The studied substances were administered to animals intravenously at a dose of 1 mg/kg at a constant velocity and constant volume. Animals of the control series were intravenously administered 1 ml of 0.9% solution of sodium chloride. The threshold of electrical cardiac fibrillation was determined 5, 10, 20, 30, 40 and 60 minutes after intravenous administration of the medicinal product.

The threshold of electrical cardiac fibrillation remained stable throughout the whole period of observation in the control experimental series. The experiment demonstrated that all animals after intravenous administration of the studied substance exhibited a statistically significant (P<0.05) increase of the electrical cardiac fibrillation threshold.

Thus, the study demonstrated that substances 1-9 possessed a pronounced antifibrillation activity.

### Effect on the Threshold of Electrical Ventricle Fibrillation in Rats in the Setting of Acute Myocardial Ischemia.

The threshold of electrical ventricle fibrillation in rats was determined by the same method as in the study of the effect of the studied substances on the threshold of electrical ventricle fibrillation in rats. Acute myocardial ischemia was caused by one-stage ligation of the left coronary artery 1-2 mm below its exit from under the atrial auricle.

During the study substances were administered intravenously at a dose of 1 mg/kg at a constant velocity and constant volume 30 minutes after ligation of the coronary artery. The threshold of electrical cardiac fibrillation was determined 5 minutes prior to ligation of the coronary artery, 25 minutes after ligation and 10, 20, 30 minutes after cessation of intravenous administration of the studied substance.

The study demonstrated that one-stage ligation of the left coronary artery caused a statistically significant (P<0.05) reduction in the threshold of electrical cardiac fibrillation. The studied substance administered 30 minutes after ligation of the coronary artery significantly (P<0.05), almost to the baseline level, increase the threshold of electrical cardiac fibrillation and thus exhibit a pronounced antifibrillation activity in the setting of myocardial ischemia.

### Effect on Left Ventricular Ejection Fraction in Intact Rats

The study was conducted in 20 anesthetized white outbred rats weighing 350-400 g. The animals were randomized into 2 groups: control (n=10) and animals, which received intravenous injections of the studied substance (n=10). The anesthetized animals were fixed supine for echocardiography. The measurements were performed in the setting of closed chest and spontaneous breathing. The measurements were made in a single dimension M-modal mode and two-dimension B-modal mode with the echocardiographic sensor in the parasternal position along the major cardiac axis. The M-modal mode was used to assess the end-systolic (ESD) and end-diastolic (EDD) dimensions of the left ventricle, then the method of Teichholz was used to calculate such heart pump function parameters as the ejection fraction (EF), shortening fraction (SF), end-systolic volume (ESV), end-diastolic volume (EDV) of left ventricle and the stroke volume of the heart. Echocardiographic parameters were evaluated using at least five consequent cardiac cycles.

The studied substance was administered to animals intravenously at a dose of 1 mg/kg at a constant velocity and constant volume. Animals of the control series were intravenously administered 1 ml of 0.9% solution of sodium chloride.

The state of intracardiac hemodynamics was assessed 10, 30, 45 and 60 minutes after i/v administration of the studied substance.

The study demonstrated that intravenous administration of substances 1-9 at a dose of 1 mg/kg did not affect the state of intracardiac hemodynamics, in particular, the contractile status of the left ventricle.

Thus, the specified agent, in contrast to the reference antiarrhythmic medicinal products of the I and III classes according to the classification of Vaughan Williams, has no negative inotropic effect, i.e., does not reduce the myocardial contractility. Comparison of antiarrhythmic and antifibrillation activity of the studied substances with Quinidine, Procainamide, Etmozin, Verapamil and Propranolol.

Several comparative experiments have been performed within the preclinical study.

### Experiment 1

The study was conducted in anesthetized white outbred rats. The objective of the experiment was to study the efficiency of intravenous administration of the studied substances and known antiarrhythmic agents in the model of aconitic arrhythmia in anesthetized rats.

The studied substances and medicinal products for comparison were administered in active doses 5-10 minutes prior to intravenous administration of aconitine.

The experiment demonstrated that substance 1-9 were superior to the known antiarrhythmic agents (Quinidine, Procainamide, Etmozin, Verapamil and Propranolol) according to the antiarrhythmic activity and range of antiarrhythmic action. Etmozin was the most efficient among the reference antiarrhythmic agents at this form of arrhythmia. Verapamil was the least effective. Lidocaine had the shortest effect.

It should be noted that intravenous use of Quinidine, Procainamide, Etmozin and

Propranolol at doses that provide for antiarrhythmic effect in 50% of rats, resulted in inhibition of the baseline heart rate and contraction, which was reflected by lengthening of intervals R−R, PQ and QT.

In contrast to the known antiarrhythmic agents, substances 1-9 at doses of 0.03-0.30 mg/kg, i.e. at doses providing for antiarrhythmic effect in 50-100% of rats had no significant effect on the sinus node function and atrioventricular conduction.

### Experiment 2

The objective of the study was to investigate the antiarrhythmic activity of the compositions under comparison in case of per os administration. The study was conducted in white outbred rats. The studied substance and medicinal products for comparison were administered to the stomach 60 minutes prior to intravenous administration of aconitine.

The study demonstrated that according to the antiarrhythmic activity and efficacy substances 1-9 were much superior to the known antiarrhythmic agents (Quinidine, Procainamide, Etmozin, Verapamil and Propranolol) when used per os. Intragastric administration of substances 1-9 in rats at doses of 0.4-1 mg/kg 60 minutes prior to intravenous administration of arrhythmogenic aconitine dose (20 μg/kg) completely prevents the development of cardiac arrhythmias in 50-100% of animals.

### Experiment 3

The objective of the study was to compare the prevention efficacy of the studied substances and the known antiarrhythmic agents (Quinidine, Procainamide, Etmozin, Verapamil and Propranolol) at the irreversible cardiac fibrillation caused by administration of aconitine at an absolutely lethal dose of 200 μg/kg (LD₁₀₀). Experiments were performed in conscious mice weighing 20-24 g. Medicinal products were administered intraperitoneally at the active doses 25-30 minutes prior to intravenous administration of aconitine. LD₅₀/ED₅₀ ratio was used as an assessment criterion for the range of therapeutic (antiarrhythmic) action.

The study demonstrated that in 80 control experiments intravenous administration of aconitine in mice at a dose of 200 μg/kg led to the development of irreversible cardiac fibrillation and the death of 100% of animals within the first 60 minutes.

Preliminary intraperitoneal administration of the studied substances in mice at doses of 0.3-0.5-1-2-5 mg/kg prior to intravenous administration of an absolutely lethal dose of aconitine prevents the irreversible cardiac fibrillation and protects from death 30-50-80-80-95% of animals, respectively.

Substance 1-9 are significantly superior to the known antiarrhythmic agents according to their antifibrillation activity, efficiency and range of antifibrillation action.

Thus, the results of comparative studies demonstrated that substances 1-9 with different methods of administration were superior to Quinidine, Procainamide, Etmozin, Verapamil and Propranolol according to the antiarrhythmic activity and range of therapeutic (antiarrhythmic) action.

Substances 1-9 at effective antiarrhythmic doses in contrast to the majority of antiarrhythmic agents do not have an inhibiting effect on the function of the sinus node and conduction.

The Impact of the Studied Substances on the Cardiac Arrhythmia Caused by Barium Chloride as Compared with Quinidine and Procainamide

The study was conducted in 60 white outbred rats. Control experiments immediately after the administration of barium chloride solution demonstrated that the general condition of rats was characterized with short fibrillary, fascicular and spastic muscle contraction, breath shortening, sudden pallor followed by cyanosis of visible mucous membranes, involuntary urination, diarrhea. Out of 15 control animals, which received barium chloride at a dose of 20 mg/kg, 60% (12/15) of the rats died.

ECG performed 30-60 seconds after barium chloride administration showed bradycardia and arrhythmia. Rhythm disturbances were mostly of a ventricular type, in the form of ventricular bigeminy, trigeminy, group extrasystoles, transient ventricular standstill, flutter and ventricular fibrillation. In addition, various types of conduction disorders and increased ST segment above contours were observed.

Intravenous administration of the studied substances at doses of 0.05-0.1 mg/kg 10 min prior to the injection of barium chloride prevented the development of arrhythmias in 60-90% of rats. Antiarrhythmic effect of the studied substances combines with antitoxic effect against lethality from barium chloride. Thus, the antitoxic effect of substances 1-9 at a dose of 0.05 mg/kg was observed in 40% of rats and at a dose of 0.1 mg/kg—in 60% of the rats.

In rats, which received a preliminary injection of the studied substances, external presentation of intoxication caused by administration of barium chloride was poor or completely absent.

In similar experimental condition Quinidine at doses of 5 mg/kg and 10 mg/kg according to its antiarrhythmic and antitoxic effect was approximately similar to that of substances 1-9 at doses of 0.05 mg/kg and 0.1 mg/kg.

In this model of cardiac arrhythmia Procainamide showed the weakest antiarrhythmic and antitoxic activity, which corresponds to the literature data.

Thus, substances 1-9 in the model of ventricular type arrhythmia caused by barium chloride are much superior to Quinidine and Procainamide according to their activity.

### Local Anesthetic Effect

The local anesthetic effect of the studied substances was investigated in 10 rabbits. The effect on terminal anesthesia was investigated by the method of Rainier-Valet, by identifying the corneal reflex to mechanical irritation every 5-10 min after instillation of 2 drops of 0.1%-0.25%-0.5% solutions of the studied substances.

The ability of the medicinal product to cause infiltration anesthesia was studied in rabbits by the method of Bullbring-Vida (pain method).

The time of anesthesia onset, its depth and duration were determined. Activity of the studied substances at topical anesthesia was compared to dicaine; at infiltration anesthesia-to novocaine.

The results demonstrated that the studied substances cause topical anesthesia.

The onset of topical anesthesia by instillation of 0.1%, 0.25% and 0.5% solutions of the studied substances into the rabbit conjunctival sac was 6.3±2.9 minutes.

The duration of the terminal anesthesia of the rabbit eye caused by a 0.1% solution of the studied substance was about 3 hours; by instillation of 0.25% and 0.5% solutions-4 hours and 6 hours, respectively.

In a similar experiment, the dicaine in concentrations of 0.1% and 0.5% causes terminal anesthesia of the rabbit eye in 1-2 min with the average duration of 37 min and 52 min, respectively. Thus, the studied substances possess a pronounced anesthetic effect. According to the anesthetic activity, the studied substances are equal to dicaine, but they are superior to dicaine according to effect duration. The studied substances are inferior to dicaine according to the anesthesia depth.

The study of substances 1-9 ability to cause infiltration anesthesia demonstrated that anesthesia in rabbits occurs 10-15 min after subcutaneous administration of 0.05% and 0.1% solutions and is characterized by an increased threshold of pain irritation, which lasts for 24-48 hours.

In the same experimental conditions, the local anesthetic effect of 0.5% solution of novocaine lasts about 90 min.

Thus, substances 1-9 have a pronounced and long-lasting local anesthetic effect.

### Effect on the Aseptic Inflammation Induced in Animals by Administration of Formalin, Histamine, Polyglucin and Serotonin

Anti-inflammatory effect of the studied substances was investigated in experiments on white outbred rats.

Aseptic inflammation was induced by subplantar administration of formalin solution (0.2 ml of 1%), histamine (0.1 ml of 0.01%), polyglucin (0.1 ml of 6%) and serotonin (0.1 ml of 0.01%) into the right rear paw of rats. The volume of paws was measured oncometrically. The studied substances were administered intraperitoneally 2-3 h prior to the induced aseptic inflammation. The anti-inflammatory effect was assessed by the difference in the volume of paws of control and experimental animals.

The experiments demonstrated that the studied substances at doses of 1-5 mg/kg possessed a significant anti-inflammatory effect, most pronounced in the model of histamine inflammation.

### Study of Anticonvulsant Activity (Antiepileptic Effect)

The studies of predicted anticonvulsant activity were performed on non-pedigree white male rats weighing 150-180. The animals of experimental groups (n=10) received single intragastric injections of the studied substances at doses of 1-5 mg/kg. Convulsive condition of the animals was simulated by a single subcutaneous injection of pentylenetetrazole (corazol) at a dose of 80 mg/kg. The test time was based on data on the anticonvulsive activity peaks of the medicinal products. Pronouncement of the anticonvulsive effect was assessed by dynamics of convulsions latent period, type and duration of convulsions in minutes and by the lethality rate. The studied substances were administered 1 hour prior to the administration of the convulsive agent. The intensity of convulsions was evaluated using a 5-point scale: 0 —no convulsive activity; 1—hyperkinesis; 2 —tremble, tremor; 3 —clonic convulsions of the forelegs, rising on hind legs; 4 —pronounced tonic-clonic convulsions with the animal falling onto its side, the presence of tonic phase of the extension; 5 —repetitive clonic-tonic convulsions, loss of posture, death. Anticonvulsant effect was defined as the protection of animals against development of clonic, tonic convulsions and lethality.

The results demonstrated that in the control group of animals after injection of corazol the duration of the latent period of convulsions amounted to an average of 5.75 min, and the duration of convulsions was 6.00 min. Convulsive disorder that developed in rats of this group was accompanied by severe tonic-clonic convulsions, which regularly repeated, with a distinct phase of tonic extension (opisthotonus). Lethality in this group was 100%. The studied substances inhibited the development of convulsions in 64% of animals, and provided for the prevention of lethality in 90% of animals.

### Study of Broncholytic Effect (COPD)

In experiments in rabbits with the COPD model of bronchial asthma (n=6), an average decreased of respiration rate 13 in 1 minute and reduction of wheezing were observed 15 minutes after administration of the studied substances. The duration of the broncholytic effect was 2 hours.

### Study of Acute Toxicity

The acute toxicity of the studied substances was investigated with a single intravenous, intraperitoneal and oral administration in mice and rats within the dose range from minimally lethal to absolutely lethal. Acute toxicity of the studied substances in different animals with different methods of administration is presented in Table 6.

TABLE 6

| | Number of | Method of | |
|---|---|---|---|
| Animal | animals | administration | LD₅₀ (mg/kg) |
| Mouse | 90 | Oral | 50.0 (42.7-64.3) |
| | | Intraperitoneal | 15.8 (11.8-19.3) |
| | | Intravenous | 6.5 (4.4-8.6) |
| Rat | 60 | Oral | 55.9 (44.3-66.9) |
| | | Intraperitoneal | 15.0 (12.4-16.8) |
| | | Intravenous | 7.0 (4.3-7.6) |

The presentation of acute poisoning in laboratory animals is characterized by weakness, adynamia, lethargy, decreased muscle tone, loss of coordination, respiratory depression. They are followed by clonic convulsions; death occurs in cases of asphyxia.

### Study of Chronic Toxicity

In the study of chronic toxicity the studied substances were administered intragastrically in rats daily for 6 months at doses of 1-10 mg/kg. Long-term administration at the mentioned doses did not cause death of animals, changes in behavior, weight gain, histomorphology of internal organs, presentation of peripheral blood and functional state of kidneys.

The macroscopic examination of internal organs of animals in the experimental and control groups revealed no visible pathological changes. Morphological examinations of the internal organs and brain tissues of animals treated with the studied substances during a specified period identified the following: uneven vascular congestion of internal organs, granular degeneration of liver, kidney and heart cells in the control group.

The organs of rats receiving the studied substances orally at a dose of 10 mg/kg (dose exceeding the therapeutic dose by 10-20 times) for 6 months were characterized by rapid swelling of the brain tissue and detachment of meninges over a large area. The heart has local, sometimes diffuse hemorrhages between muscle fibers; the liver was with destruction and micronecrosis of the parenchyma; kidneys were characterized by the presentation of necrotic nephrosis; the gastrointestinal tract had foci of hemorrhage in the submucosal and muscle layers, dystonia and plasmorrhagia the blood vessels walls.

### EXAMPLE 3.

A pharmaceutical composition for oral administration was obtained, which has a form of a flat round tablet; the tablet composition is presented in Table 7.

TABLE 7

| | Quantity of the active ingredient |
|---|---|
| | and excipients, mg per 1 tablet |
| Claimed drug | 25 |
| Sucrose | 65.5 |
| Potato starch | 7.5 |
| Calcium stearate | 1 |
| Sodium croscarmellose | 1 |

The studies have demonstrated that the specified agent possesses antiarrhythmic activity in models reproducing various cardiac arrhythmias, including the most life-threatening one-fibrillation.

Antiarrhythmic properties of the specified agent were studied in aconitic and barium chloride models of cardiac arrhythmia. A number of known antiarrhythmic agents were used for comparison. The studies have demonstrated that the specified agent possesses a high antiarrhythmic activity in models reproducing various cardiac arrhythmias, including the most life-threatening one-ventricle fibrillation and myocardial contractility. Thus, the specified agent possesses a pronounced high efficiency in both arrhythmia models but in contrast to the reference antiarrhythmic medicinal products of the I and III classes according to the classification of Vaughan Williams, has no negative inotropic effect, i.e., does not reduce the myocardial contractility.

The studies have also demonstrated that the specified agent in an animal experiment with a COPD model possesses broncholytic properties, and as a consequence, can be considered as a potential means of therapy in patients with the COPD and accompanying arrhythmias. The study demonstrated that the specified agent also possesses an antiepileptic effect, anti-inflammatory effect, pronounced and long-lasting local anesthetic action.

### EXAMPLE 4

### A Determination Procedure for Pharmaceutical Substance Components

The pharmaceutical substance composition is analyzed by means of high-performance liquid chromatography on any type of a liquid chromatograph equipped with a UV detector and a mass detector.
Required Instruments, Reagents and Materials.

A liquid chromatograph of any type;

A chromatographic system used for analysis should contain at least the following components:

One high-pressure pump, a flow-through degasser for mobile phase, an automatic sampling device, a chromatographic column, and a UV detector. Availability of a second pump (for column washing), a thermostat for analyses under constant temperature, and a mass detector for component identification.

Analytical chromatographic column ReproSil-Pur Basic C18, 5 μm, 250*4.6 mm, manufactured by Dr. Maisch GmbH, with a precolumn;
A pH meter with measurement accuracy to hundredths of pH units, calibrated;
A magnetic mixer with a stir bar;
Glass volumetric cylinders and beakers for preparation of solutions;
Capped glass bottles (dark) for storage of solutions;
50 mL volumetric flasks to prepare solutions of the analyzed substances;
High-quality acetonitrile, e. g., Acetonitrile for UHPLC Supergradient from
Panreac, or its analog of similar quality;
High-purity water suitable for work in UHPLC;
Ammonium formate, pharmacopoeial grade;
Formic acid, pharmacopoeial grade (as approx. 1% aqueous solution);
Trifluoroacetic acid.

### Preparation of a Chromatographic System for Analysis.

To prepare a chromatographic system for analysis, take fresh mobile phase and carefully fill it into a hydraulic line up to the column. Flushing with mobile phase at 5 mL/min during 30 minutes is recommended. After this, equilibrate the rest of the hydraulic line (the column, capillaries and detector) with mobile phase. Mobile phase should be flushed through the entire hydraulic system at 1 mL/min during 120 minutes. Then, if a drift of a base line on a chromatogram is less than 10 mAU within 10 minutes, the system is ready for analyses.

### Injection Volume Determination.

An optimal injection volume should be determined for each individual device, because the quality of obtained chromatograms (peak heights and widths) depends on the injection volume and geometrical parameters of a detector cell. The optimal injection volume determined experimentally should correspond to the main component peak height from 300 to 1,000 mAU and the heights of the relevant impurity peaks from 20 to 200 mAU. These values are not strict and are provided as recommended ones. The injection volume is assumed to fall within the range from 2 to 10 μL. The average value of this parameter, 5 μL, is specified hereinafter.

Procedure of Analysis.

5 μL of the test solution and 5 μL of the reference solution are alternately chromatographed in a liquid chromatograph to obtain at least 2 chromatograms for each solution under the following conditions:
analytical chromatographic column ReproSil-Pur Basic C18, 5 μm, 250*4.6 mm, manufactured by Dr. Maisch GmbH;
mobile phase prepared according to the requirements of the section “Mobile phase preparation” (see below), using 1 volume of acetonitrile and 3 volumes of ammonium formate buffer solution (the preparation is described below);
elution in an isocratic mode;
mobile phase flow of 1.0 mL/min;
integration time: 40 min (analysis cycle duration);
detection at 220 nm wavelength;
the detector's sensitivity is determined experimentally.

### Column Flushing.

After each 8-12 cycles, the column needs flushing, as can be noticed from increasing pressure level.

Flushing is performed with a flushing solution during at least 60 minutes at the speed of 1 mL/min. The column is considered flushed if 60 minutes after the flushing the base line does not deviate from a “zero value” by more than 10 mAU. After flushing, “Preparation of a Chromatographic System for Analysis” (see above) procedure is necessary to carry out the analyses.

### Notes.

### Preparation of Ammonium Formate Buffering Solution for Mobile Phase.

Weigh 200 mg (a precisely weighed amount) of ammonium formate in a glass beaker on an analytical balance with precision up to 0.2 mg. Measure 1,000 mL of pure water using a volumetric cylinder. Use these 1,000 mL of water to completely wash the weighed ammonium formate from the glass beaker for weighing into a vessel for preparing the buffer solution. Place a magnetic stir bar into the vessel for preparing the buffer solution. Place the vessel on a magnetic mixer.

Stir 3-5 minutes to completely dissolve ammonium formate, then submerge a pH meter electrode into the vessel, and measure the pH level. Bring the pH level to 5.00 using 1% formic acid solution. When the solution with the required pH is prepared, pour the beaker's contents into a 1 L dark glass bottle with a seal cap. The prepared ammonium formate buffer solution is suitable for use during 24 hours if stored in a dark place at room temperature.

### Mobile Phase Preparation.

Pour 100 mL of acetonitrile into a clean, dry 500 mL volumetric cylinder. After this, pour acetonitrile from the cylinder (as completely as possible) into a vessel for preparing the mobile phase. Then pour into the same cylinder 300 mL of ammonium formate buffer solution for preparing the mobile phase. Pour the buffer solution from the cylinder (as completely as possible) into the vessel for preparing the mobile phase. Mix the contents of the vessel for preparing the mobile phase. The prepared mobile phase is poured into a dark glass bottle with a seal cap. The mobile phase is suitable for use during 24 hours. Preparation of substance solution with identified impurities (reference solution).

Place about 50 mg (a precisely weighed amount) of the substance with identified (by LCMS method or using external standards) impurities into a 50 mL volumetric flask and dissolve in the mobile phase, make up to the mark with the same solvent, and stir.

### Preparation of the Test Substance Solution (Test Solution).

Place about 50 mg (a precisely weighed amount) of the test substance into a 50 mL volumetric flask and dissolve in the mobile phase, make up to the mark with the same solvent, and stir.

### Preparation of the Flushing Solution.

Add 500 μL of trifluoroacetic acid to 500 mL of acetonitrile, and stir.

Pour into a dark glass bottle. The flushing solution is suitable for use during 1 month after preparation.

A procedure for Determination of the Pharmaceutical Substance Components in the Finished Dosage Form.

This procedure has been developed to determine the pharmaceutical substance components in the finished tablet dosage form containing 25 mg of the pharmaceutical substance in each tablet. The analysis is carried out by means of high-performance liquid chromatography using any type of a liquid chromatograph equipped with a UV detector and a mass detector. This procedure is based on the pharmaceutical substance component determination procedure, with addition of the required sample preparation procedure.

### Required Instruments, Reagents and Materials.

A liquid chromatograph of any type;

A chromatographic system used for analysis should contain the following components:

One high pressure pump, a flow-through degasser for mobile phase, an automatic sampling device, a chromatographic column, and a UV detector. Availability of a second pump (for column washing), a thermostat for analyses under constant temperature, and a mass detector for component identification.
Analytical chromatographic column ReproSil-Pur Basic C18, 5 μm, 250*4.6 mm, manufactured by Dr. Maisch GmbH, with a precolumn;
A pH meter with measurement accuracy to hundredths of pH units, calibrated;
A magnetic mixer with a stir bar;
An ultrasonic bath of any type;
A laboratory centrifuge creating an acceleration of at least 15-18 g.
Glass volumetric cylinders and beakers for preparation of solutions;
Capped glass bottles (dark) for storage of solutions;
25 mL capped volumetric flasks to prepare solutions of the analyzed samples;
100 μL and 1,000 μL automatic pipettes (calibrated) with nozzles;
1.5 mL and 2.0 mL single-use capped plastic test tubes;
High-quality acetonitrile, e. g., Acetonitrile for UHPLC Supergradient from Panreac, or its analog of similar quality;
High-purity water suitable for work in UHPLC;
Ammonium formate, pharmacopoeial grade;
Formic acid, pharmacopoeial grade (as approx. 1% aqueous solution);
Trifluoroacetic acid.

### Sample Preparation.

To prepare a sample for analysis, place one tablet containing 20 to 30 mg of the claimed pharmaceutical substance into a 25 mL volumetric flask. Then pour 15-20 mL of the mobile phase into the flask, and place the flask into an active ultrasonic bath for 7-10 minutes. After the ultrasonic treatment, the flask should contain a homogeneous white suspension without visible fragments of the tablet being dissolved. Then make up the flask to the mark with the mobile phase, stopper the flask and shake several times for complete mixing.

After that, place 1 mL of the suspension into a single-use plastic test tube using an automatic measuring pipette. Close the test tube with a cap, and place it into a balanced centrifuge. Then centrifuge the test tube with the suspension during 7-10 minutes at the acceleration of 15-18 g. The test tube after the centrifuging should contain a small amount of a white precipitate with a colorless solution above it. Place 500 μL of the colorless solution into a chromatographic vial, using an automatic measuring pipette. This should be made carefully to prevent the precipitate from entering the sample volume.

### Preparation of a Chromatographic System for Analysis.

To prepare a chromatographic system for analysis, take fresh mobile phase and carefully fill it into a hydraulic line up to the column. Flushing with mobile phase at 5 mL/min during 30 minutes is recommended. After this, equilibrate the rest of the hydraulic line (the column, capillaries and detector) with mobile phase. Mobile phase is recommended to be flushed through the entire hydraulic system at 1 mL/min during 150 minutes. Then, if a drift of a base line on a chromatogram is less than 10 mAU within 10 minutes, the system is ready for analyses.

### Injection Volume Determination.

An optimal injection volume should be determined for each individual device, because the quality of obtained chromatograms (peak heights and widths) depends on the injection volume and geometrical parameters of a detector cell. The optimal injection volume determined experimentally should correspond to the main component peak height from 300 to 1,000 mAU and the heights of the relevant impurity peaks from 20 to 200 mAU. These values are not strict and are provided as recommended ones. The injection volume is assumed to fall within the range from 2 to 10 μL. The average value of this parameter, 5 μL, is specified hereinafter.

### Procedure of Analysis.

5 L of the test sample solution and 5 μL of the reference solution are alternately chromatographed in a liquid chromatograph to obtain at least 2 chromatograms for each solution under the following conditions:
analytical chromatographic column ReproSil-Pur Basic C18, 5 μm, 250*4.6 mm, manufactured by Dr. Maisch GmbH;
mobile phase prepared according to the requirements of the section “Mobile phase preparation” (see below), using 1 volume of acetonitrile and 3 volumes of ammonium formate buffer solution (the preparation is described below);
elution in an isocratic mode;
mobile phase flow of 1.0 mL/min;
integration time: 40 min (analysis cycle duration);
detection at 220 nm wavelength;
the detector's sensitivity is determined experimentally.

### Column Flushing.

After each 12-15 cycles, the column needs flushing, as can be noticed from increasing pressure level.

Flushing is performed with a flushing solution during at least 60 minutes at the speed of 1 mL/min. The column is considered flushed if 60 minutes after the flushing the base line does not deviate from a “zero value” by more than 10 mAU. After flushing, the chromatographic system should be prepared for analysis (see above).

### Notes.

### Preparation of Ammonium Formate Buffer Solution for Mobile Phase.

Weigh 200 mg (a precisely weighed amount) of ammonium formate in a glass beaker on an analytical balance with precision up to 0.2 mg. Measure 1,000 mL of pure water using a volumetric cylinder. Use these 1,000 mL of water to completely wash the weighed ammonium formate from the glass beaker for weighing into a vessel for preparing the buffer solution. Place a magnetic stir bar into the vessel for preparing the buffer solution. Place the vessel on a magnetic mixer.

Stir 3-5 minutes to completely dissolve ammonium formate, then submerge a pH meter electrode into the vessel, and measure the pH level. Bring the pH level to 5.00 using 1% formic acid solution. When the solution with the required pH is prepared, pour the beaker's contents into a 1 L dark glass bottle with a seal cap. The prepared ammonium formate buffer solution is suitable for use during 24 hours if stored in a dark place at room temperature.

### Mobile Phase Preparation.

Pour 100 mL of acetonitrile into a clean, dry 500 mL volumetric cylinder. After this, pour acetonitrile from the cylinder (as completely as possible) into a vessel for preparing the mobile phase. Then pour into the same cylinder 300 mL of ammonium formate buffer solution for preparing the mobile phase. Pour the buffer solution from the cylinder (as completely as possible) into the vessel for preparing the mobile phase. Mix the contents of the vessel for preparing the mobile phase. The prepared mobile phase is poured into a 1 L dark glass bottle with a seal cap. The mobile phase is suitable for use during 24 hours.

PReparation of Substance Solution with Identified Impurities (Reference Solution).

Place about 25 mg (a precisely weighed amount) of the substance with identified components into a 25 mL volumetric flask and dissolve in the mobile phase, make up to the mark with the mobile phase, and stir.

### Preparation of the Test Sample Solution.

Preparation of this solution is described in “Sample Preparation” section

Preparation of the Flushing Solution.

Add 500 μL of trifluoroacetic acid to 500 mL of acetonitrile, and stir.

Pour into a dark glass bottle. The flushing solution is suitable for use during 1 month after preparation.

The result of the present invention is the creation of a more efficient and safe cardioprotective agent and a pharmaceutical composition based on this agent for the prevention and treatment of arrhythmia, possessing the expanded possibilities of prevention of potentially malignant and malignant ventricular arrhythmias, ventricular fibrillation and reduction of the ejection fraction (index of myocardial contractility), which is the immediate cause of a sudden cardiac death, as well as new functionalities of the complex treatment of arrhythmia, complicated by the chronic obstructive pulmonary disease (COPD) and epilepsy, as well as expansion the range of cardioprotective agents that possess a high antiarrhythmic activity in various forms of arrhythmia.

What is claimed is:

1. An antiarrhythmic agent obtained from Aconitum plants (wolfsbane) of Ranunculaceae family (buttercup family) contains alkaloids lappaconitine, N-acetylsepaconitine, 1-desmethyllappaconitine, ranaconitine, N-desacetyllappaconitine, isolappaconitine, 9-deoxylappaconitine or their pharmaceutically acceptable salts.

2. The agent according to claim 1, further comprising alkaloids in a form of hydrobromides of lappaconitine, N-acetylsepaconitine, 1-desmethyllappaconitine, ranaconitine, N-desacetyllappaconitine, isolappaconitine, 9-deoxylappaconitine.

3. The agent according to claim 1, being extracted from a rootstock of northern wolfsbane (high wolfsbane)-Aconitum septentrionale Koelle, buttercup family-Ranunculaceae.

4. The agent according to claim 1, being extracted from a rootstock of Aconitum leucostomum, buttercup family-Ranunculaceae.

5. The agent according to claim 1, being extracted from a herbage of Aconitum leucostomum, buttercup family-Ranunculaceae.

6. An oral pharmaceutical composition containing the agent according to claim 1 in an effective quantity and a pharmaceutically acceptable carrier.

7. The pharmaceutical composition according to claim 6, wherein the pharmaceutically acceptable carrier contains starch, sucrose, and calcium stearate.

8. The pharmaceutical composition according to claim 7, wherein the pharmaceutically acceptable carrier additionally contains sodium croscarmellose.

9. The agent according to claim 2, being extracted from a rootstock of northern wolfsbane (high wolfsbane)-Aconitum septentrionale Koelle, buttercup family-Ranunculaceae.

10. The agent according to claim 2, being extracted from a rootstock of northern wolfsbane (high wolfsbane)-Aconitum septentrionale Koelle, buttercup family-Ranunculaceae.

11. The agent according to claim 2, being extracted from a herbage of Aconitum leucostomum, buttercup family-Ranunculaceae.

12. An oral pharmaceutical composition containing the agent according to claim 2 in an effective quantity and a pharmaceutically acceptable carrier.

13. An oral pharmaceutical composition containing the agent according to claim 3 in an effective quantity and a pharmaceutically acceptable carrier.

14. An oral pharmaceutical composition containing the agent according to claim 4 in an effective quantity and a pharmaceutically acceptable carrier.

15. An oral pharmaceutical composition containing the agent according to claim 5 in an effective quantity and a pharmaceutically acceptable carrier.
